SEQUENCE LISTING (12 sequences)

SEQ ID NO: 1 - DNA, 19 bp, Artificial sequence
  PCR primer
ctgggctacactgagcacc

SEQ ID NO: 2 - DNA, 21 bp, Artificial sequence
  PCR primer
aagtggtcgttgagggcaatg

SEQ ID NO: 3 - DNA, 18 bp, Artificial sequence
  PCR primer
ggactgcgcttggctgcg

SEQ ID NO: 4 - DNA, 21 bp, Artificial sequence
  PCR primer
gtcggaagcagaggtcaggca

SEQ ID NO: 5 - DNA, 20 bp, Artificial sequence
  PCR primer
aagagttgggctctgtcagc

SEQ ID NO: 6 - DNA, 20 bp, Artificial sequence
  PCR primer
gactcgctccgttcctcttc

SEQ ID NO: 7 - DNA, 84 bp, Artificial sequence
  Telomere standard
ttagggttagggttagggttagggttagggttagggttagggttagggttagggttagggttagggttagggttagggttaggg

SEQ ID NO: 8 - DNA, 75 bp, Homo sapiens
cagcaagtgggaaggtgtaatccgtctccacagacaaggccaggactcgtttgtacccgttgatgatagaatggg

SEQ ID NO: 9 - DNA, 39 bp, Artificial sequence
  PCR primer
cggtttgtttgggtttgggtttgggtttgggtttgggtt

SEQ ID NO: 10 - DNA, 39 bp, Artificial sequence
  PCR primer
ggcttgccttacccttacccttacccttacccttaccct

SEQ ID NO: 11 - DNA, 23 bp, Artificial sequence
  PCR primer
cagcaagtgggaaggtgtaatcc

SEQ ID NO: 12 - DNA, 25 bp, Artificial sequence
  PCR primer
cccattctatcatcaacgggtacaa

(19) United States
(12) Patent Application Publication
(10) Pub. No.: US 2019/0160131 A1
(43) Pub. Date: May 30, 2019

(54) METHOD FOR CELL REJUVENATION USING ROSA ROXBURGHII FRUIT EXTRACTS

(71) Applicant: TCI CO., LTD, Taipei (TW)

(72) Inventors: Yung-Hsiang Lin, Taipei (TW); Yu-Hung Su, Taipei (TW)

(21) Appl. No.: 16/100,956

(22) Filed: Aug. 10, 2018

Related U.S. Application Data
(60) Provisional application No. 62/591,604, filed on Nov. 28, 2017.

(30) Foreign Application Priority Data
Apr. 30, 2018 (TW) 107114734

(51) Int. Cl. A61K 36/738; A61P 43/00

(52) U.S. Cl. CPC A61K 36/738; A61K 2236/331; A61K 2236/51; A61P 43/00

(57) ABSTRACT

The present invention provides a method for cell rejuvenation, including contacting cells with a composition containing a Rosa roxburghii fruit extract obtained by extraction of a Rosa roxburghii fruit using water, alcohol, or mixtures of water and alcohol as solvents. Said extract improves DNA repair, reduces cell damage caused by oxidative stress or inflammation, maintains the normal functions of genes, proteins, and mitochondria, facilitates telomerase reaction and reduces telomere damage by chemicals or UV radiation, thereby improving cell rejuvenation.

### CROSS-REFERENCE TO RELATED APPLICATION

This application claims priority of U.S. Provisional Application No. 62/591,604, filed on Nov. 28, 2017, and Taiwan patent application No. 107114734, filed on Apr. 30, 2018, the content of which is incorporated herein in its entirety by reference.

### BACKGROUND OF THE INVENTION

### 1. Field of the Invention

The present invention relates to a method for health care by using Rosa roxburghii fruit extracts. Particularly, the present invention relates to a method for cell rejuvenation by using Rosa roxburghii fruit extracts.

### 2. The Prior Art

With an increase in the average age of population, it has become one of the issues attracting public attention to have healthy and high-standard living of the aged, and scientific research on aging has become more and more important. There are numerous reasons for aging in individuals. The possible internal mechanisms at the molecular and cellular levels include telomere shortening at the end of chromosomes, gene mutation, poor protein synthesis efficiency, reduced mitochondrial function, and variation in cell morphology. Moreover, free radicals (such as reactive oxygen species) in the cell environment that are induced by physical or chemical factors would damage the chromosomal deoxyribonucleic acid (DNA), proteins, and lipid biomembrane. In response to such cell damage or invasion of microorganisms, inflammation is initiated in individuals. Once it turns into chronic inflammation, cell damage is exacerbated, thereby accelerating the aging of cells and individuals.

Methods for combating aging include uptake of natural antioxidants from the diet to enhance individual's ability to remove free radicals and inhibition of chronic inflammation in the body by anti-inflammatory drugs. In view of the complicating causes of aging such as those described above, any anti-aging strategy for a single target is clearly ineffective. Though the emerging stem cell therapy is highly potential in anti-aging, it is still in the development stage and the cost of treatment is high. Therefore, it is of necessity to develop a simple and novel composition for individuals or cells to stay in a youth's condition through multiple mechanisms.

### SUMMARY OF THE INVENTION

Accordingly, the present invention provides a method for cell rejuvenation, including contacting a cell with a composition containing an effective amount of a Rosa roxburghii fruit extract obtained by extraction of a Rosa roxburghii fruit with a solvent.

In one embodiment of the present invention, the solvent is water, alcohol, or a mixture of alcohol and water. The weight ratio of the solvent to the Rosa roxburghii fruit ranges from 20:1 to 1:1, and the extraction is performed at a temperature between 50° C. and 100° C.

In one embodiment of the present invention, the composition contains a water extract of Rosa roxburghii fruit at a concentration of at least 0.5 mg/mL.

In one embodiment of the present invention, the Rosa roxburghii fruit extract promotes telomerase synthesis and enhances the gene expression of telomerase reverse transcriptase (TERT), telomerase ribonucleic acid component (telomerase RNA component, TERC), or combinations thereof.

In one embodiment of the present invention, the Rosa roxburghii fruit extract inhibits chemical or UV irradiation-induced shortening of chromosomal telomere length.

In one embodiment of the present invention, the Rosa roxburghii fruit extract improves gene repair and enhances gene expression of a DNA repair protein selected from the group consisting of N-methylpurine DNA glycosylase (MPG), excision repair cross complementing 6 (ERCC6), X-ray repair cross complementing 5 (XRCC5), and any combination thereof.

In one embodiment of the present invention, the Rosa roxburghii fruit extract promotes the synthesis of chaperonin containing TCP1 complex (CCT), and enhances the gene expression of a CCT subunit selected from the group consisting of CCT2, CCT5, CCT6A, CCT7, CCT8, and any combination thereof.

In one embodiment of the present invention, the Rosa roxburghii fruit extract enhances mitochondrial activity, and inhibits the gene expression of a poly (adenosine diphosphate-ribose) polymerase (also known as poly (ADP-ribose) polymerase, PARP) selected from the group consisting of PARP1, PARP3, PARP4, PARP8, PARP11, and any combination thereof.

The present invention discloses, based on the results of gene expression analysis, that the Rosa roxburghii fruit extract reduces cell damage caused by oxidative stress or inflammation, maintains the normal functions of genes, proteins, and mitochondria, facilitates telomerase reaction and reduces telomere damage by chemicals or UV radiation, thereby improving cell rejuvenation. Thus, the present invention provides a method for cell rejuvenation by contacting cells with a composition containing the Rosa roxburghii fruit extract. The composition is in the form of powders, granules, liquid, gel or paste, and is produced as foods, beverages, nutritional supplements, pharmaceuticals, or reagents that may be administered to a human subject via an oral or topical route.

The present invention is further explained in the following examples, in reference to the accompanying drawings. It should be understood that the examples given below do not limit the scope of the invention, and that modifications can be made without departing from the scope of the appended claims.

### BRIEF DESCRIPTION OF THE DRAWINGS

FIG. 1 shows the relative expression of superoxide dismutase 2 (SOD2) gene in lipopolysaccharide (LPS)-stimulated THP-1 cells treated for 24 hours with or without a Rosa roxburghii fruit extract as compared to the expression in control cells;

FIG. 2 shows the relative expression of C-C motif chemokine ligand 3 (CCL3) gene in LPS-stimulated THP-1 cells treated for 6 hours with or without the Rosa roxburghii fruit extract as compared to the expression in control cells;

FIG. 3 shows the relative expression of interleukin-1 receptor antagonist (IL-1RA) and interleukin-10 (IL-10) genes in LPS-stimulated THP-1 cells treated for 6 hours with or without the Rosa roxburghii fruit extract as compared to the expression in control cells;

FIG. 4 shows the relative expression of MPG, ERCC6, and XRCC5 genes in LPS-stimulated THP-1 cells treated for 6 hours with or without the Rosa roxburghii fruit extract as compared to the expression in control cells;

FIG. 5 shows the relative expression of CCT2, CCT5, CCT6A, CCT7, and CCT8 genes in THP-1 cells treated with the Rosa roxburghii fruit extract for 6 or 24 hours as compared to the expression in control cells;

FIG. 6 shows the relative expression of the PARP1, PARP3, PARP4, PARP8, and PARP11 genes in THP-1 cells treated with the Rosa roxburghii fruit extract for 6 or 24 hours as compared to the expression in control cells;

FIG. 7 shows the relative expression of TERT and TERC genes in THP-1 cells treated with the Rosa roxburghii fruit extract for 6 hours or in control cells;

FIG. 8 shows the inhibition of chemical-induced telomere shortening in skin cells by a Rosa roxburghii fruit water extract; and

FIG. 9 shows the inhibition of ultraviolet irradiation-induced telomere shortening in skin cells by the Rosa roxburghii fruit water extract.

### DETAILED DESCRIPTION OF THE PREFERRED EMBODIMENT

The present invention provides a method for cell rejuvenation, including contacting cells with a composition containing a Rosa roxburghii fruit extract. The Rosa roxburghii fruit extract is obtained by extracting the fruit of Rosa roxburghii with a solvent, wherein the solvent is water, alcohol, or a mixture of alcohol and water, the weight ratio of the solvent to the Rosa roxburghii fruit ranges from 20:1 to 1:1, and the extraction is performed a temperature between 50° C. and 100° C. By gene expression analysis techniques, the Rosa roxburghii fruit extract was shown to enhance the expression of antioxidant genes and genes of anti-inflammatory cytokines, DNA repair proteins, chaperonin containing TCP1 complex (CCT) subunits, and telomerase reaction. The Rosa roxburghii fruit extract was also shown to inhibit the expression of genes associated with inflammatory reactions and mitochondrial dysfunction and to reduce chromosomal telomere shortening induced by chemicals or UV irradiation.

### Definition

Numerical quantities provided herein are approximated, experimental values that may vary within 20 percent, preferably within 10 percent, and most preferably within 5 percent. Thus, the terms “about” and “approximately” refer to within 20 percent, preferably within 10 percent, and most preferably within 5 percent of a given value or range.

The term “cell rejuvenation” as used herein refers to the process of reversing cell aging, which may be determined by many biological indicators, such as the enhanced expression of genes associated with cellular DNA repair, telomerase reaction, protein's normal function, and mitochondrial activity, and the promotion of chromosomal telomere elongation.

### Materials and Methods

### Cell Culture

Cells used in the following examples include human monocytic cell line THP-1 (ATCC TIB202), purchased from the American Type Culture Collection (ATCC), and human skin fibroblasts CCD-966SK (BCRC 60153), purchased from the Bioresource Collection and Research Center (BCRC) of Food Industry Research and Development Institute. THP-1 cells were cultured at 37° C. under 5% carbon dioxide in RPMI cell culture medium, which was prepared by supplementing RPMI medium 1640 (Gibco) with 10% fetal bovine serum (FBS) and 1% penicillin-streptomycin. CCD-966SK cells were cultured at 37° C. under 5% carbon dioxide in MEM cell culture medium, which was prepared by supplementing Minimal Essential Medium (MEM; Gibco) with 10% FBS and 1% Penicillin-Streptomycin.

### Whole Genome Microarray Analysis

The whole genome expression profiles of cells were determined using a human whole genome microarray (manufactured by Phalanx Biotech Co., Ltd.), following the steps briefly described below. According to the manufacturer's instructions, RNA was isolated from THP-1 cells with RNA Extraction Kit (Geneaid) and used as a template for reverse transcription at 37° C. and synthesis of a single-strand complementary DNA (cDNA) using an oligo dT primer including the sequence of T7 promoter. A double-strand cDNA was then synthesized using DNA polymerase and RNase H. The double-stranded cDNA, after being purified, was used as a template in an in vitro transcription reaction, which was carried out with amino allyl UTP (aaUTP) and the Amino Allyl MessageAmp™ aRNA Amplification kit (Invitrogen) to synthesize aminoallyl-modified RNA (aRNA). The purified aRNA was conjugated with an amine-reactive fluorescent dye such as Cy5 (Cy5 NHS ester; AAT Bioquest) to generate dye-labeled RNA. After removal of unbound dye by dialysis, the purified dye-labeled RNA was added to the human whole genome microarray loaded with a plurality of specific gene probes, and a hybridization reaction was performed with a hybridization kit (Phalanx). The proteins encoded by the plurality of specific genes include SOD2, which relates to antioxidant activity, CCL3, which relates to inflammatory response, anti-inflammatory cytokines such as IL-1RA and IL-10, DNA repair proteins such as MPG, ERCC6, and XRCC5, multiple subunits of chaperonin containing TCP1 complex (CCT) such as CCT2, CCT5, CCT6A, CCT7, and CCT8, and various poly (ADP-ribose) polymerases (PARPs) that are associated with mitochondrial dysfunction, including PARP1, PARP3, PARP4, PARP8, and PARP11. The fluorescence signal indicating hybridization was detected by Agilent microarray scanner after the hybridization reaction. The raw data were normalized and the relative gene expression was expressed as log 2 value. For statistical analysis, standard deviation was calculated based on the relative expression of each gene using the STDEV function in Excel, and statistical difference was determined using the single-tailed Student's T-test (TTEST).

### Gene Expression Analysis

Cellular gene expression of telomerase reverse transcriptase (TERT) and telomerase RNA component (TERC), both involved in telomerase reaction, was measured based on quantitative polymerase chain reaction (qPCR), following the steps briefly described below. According to the manufacturer's instructions, RNA was isolated from cells with RNA Extraction Kit (Geneaid), and 2000 ng of the RNA was reverse transcribed into cDNA at 37° C. using SuperScript® III Reverse Transcriptase (Invitrogen). Thereafter, the cDNA was subjected to qPCR to obtain melting curves. The qPCR was performed with a PCR thermocycler (Step One Plus Real-Time PCR system; Applied Biosystems) using KAPA CYBR FAST qPCR Kit (2×) (KAPA Biosystems) and primers of target genes such as TERT and TERC and the internal control of glyceraldehyde 3-phosphate dehydrogenase (GAPDH) gene (Table 1).

TABLE 1

| | Nucleotide sequences of forward (F) and |
|---|---|
| Gene | reverse (R) primers |
| GAPDH | F: CTGGGCTACACTGAGCACC (SEQ ID NO: 1) |
| | R: AAGTGGTCGTTGAGGGCAATG (SEQ ID NO: 2) |
| TERT | F: GGACTGCGCTTGGCTGCG (SEQ ID NO: 3) |
| | R: GTCGGAAGCAGAGGTCAGGCA (SEQ ID NO: 4) |
| TERC | F: AAGAGTTGGGCTCTGTCAGC (SEQ ID NO: 5) |
| | R: GACTCGCTCCGTTCCTCTTC (SEQ ID NO: 6) |

Lastly, the 2^{-ΔΔCT} method was used to determine the relative expression of target genes. The cycle threshold (C_{T}) value of GAPDH gene was used as the cycle threshold value of reference gene (internal control). The fold change was calculated according to the following formula:

ΔC_{T}=C_{T} of target gene in experimental or control group−C_{T} of internal control

ΔΔC_{T}=ΔC_{T} of the experimental group−ΔC_{T} of the control group

Fold change=2^{-ΔΔCt mean}

For statistical analysis, standard deviation was calculated based on the relative expression of each gene using the STDEV function in Excel, and statistical difference was determined using the single-tailed Student's T-test (TTEST).

### Telomere Length Determination

Telomere length is determined based on qPCR technique. First, genomic DNA samples were extracted from cells. Next, the genomic DNA samples (20 ng) were subjected to qPCR to obtain melting curves. The qPCR was performed with a PCR thermocycler using KAPA CYBR FAST qPCR Kit (2×) (KAPA Biosystems) and primers of the telomere and the single-copy 36B4 gene (Table 2). In addition, serially diluted standards of the telomere (10- to 10⁶-fold dilutions starting from 60 pg) and the 36B4 gene (10- to 10⁶-fold dilutions starting from 200 pg) were prepared and subjected to qPCR under the following reaction conditions to obtain standard curves (Ct/kb): a denaturation phase at 95° C. for 10 minutes followed by 40 cycles of denaturation (95° C.) for 15 seconds and annealing (60° C.) for 1 minute. The telomere standard was an oligonucleotide having 14 repeats of TTAGGG (SEQ ID NO: 7); the standard of 36B4 gene had the nucleotide sequence of SEQ ID NO:8. Lastly, the average length of telomere in the genomic DNA samples was determined from the aforementioned standard curves and the cycle threshold values of telomere and 36B4 gene in the genomic DNA samples.

TABLE 2

| Target | Nucleotide sequences of forward (F) and |
|---|---|
| DNA | reverse (R) primers |
| Telomere | F: CGGTTTGTTTGGGTTTGGGTTTGGGTTTGGGTTTGGG |
| | TT (SEQ ID NO: 9) |
| | R: GGCTTGCCTTACCCTTACCCTTACCCTTACCCTTACC |
| | CT (SEQ ID NO: 10) |
| 36B4 | F: CAGCAAGTGGGAAGGTGTAATCC |
| | (SEQ ID NO: 11) |
| | R: CCCATTCTATCATCAACGGGTACAA |
| | (SEQ ID NO: 12) |

### Example 1

Preparation of Rosa roxburghii Fruit Extract

First, the whole fruit of Rosa roxburghii including the peel and the thorn is washed, dried, and coarsely ground using a homogenizer. Next, the Rosa roxburghii fruit homogenate is extracted using water, alcohol, or a mixture of water and alcohol as the solvent. The weight ratio of the solvent to the Rosa roxburghii fruit homogenate ranges from 20:1 to 1:1. The extraction temperature is between 50° C. to 100° C., preferably between 70° C. and 95° C. In one embodiment, the time for extraction is from 0.5 to 3 hours.

After the abovementioned extraction, the Rosa roxburghii fruit extract is cooled to room temperature. The extract may be filtered through a 400 mesh filter to remove residual solids. The filtered Rosa roxburghii fruit extract may further be concentrated under reduced pressure (less than 1 atm) at 45° C. to 70° C. to obtain a concentrated product. In order to produce a solid form of Rosa roxburghii fruit extract, the concentrated product of Rosa roxburghii fruit extract may be subjected to spray drying to remove the solvent, thereby obtaining the powder of Rosa roxburghii fruit extract. Before the step of spray drying, the Rosa roxburghii fruit extract may be optionally mixed with maltodextrin at a weight ratio (w/w) ranging from 20:1 to 1:1.

### Example 2

Reduction of Cell Damage, Improvement of DNA Repair, and Promotion of Cell Rejuvenation by Treatment of Rosa roxburghii Fruit Extract

The change in genomic expression pattern was analyzed in human monocytic cell line THP-1 treated with a Rosa roxburghii fruit water extract using a human whole genome microarray. The same analysis was also performed in cells treated with the Rosa roxburghii fruit extract prepared with alcohol or a mixture of alcohol and water (data not shown). First, THP-1 cells were seeded at 1.5×10⁵ cells/well in a 6-well plate, where each well contained 2 mL of RPMI cell culture medium. After culture at 37° C. for 24 hours, the cell culture medium was removed and the cells were washed with PBS. Then, cells in each well were treated with 500 μL of 1 mg/mL Rosa roxburghii fruit water extract and 500 μL of RPMI cell culture medium without FBS (experimental group), or treated only with 500 μL of RPMI cell culture medium without FBS (control group). The cells of experimental group were subjected to gene expression analysis after incubated at 37° C. for 6 or 24 hours with or without the stimulus of 10 μg/mL lipopolysaccharide (LPS). The relative expression of indicated genes was the fold change compared to the expression of same gene in the control cells after the same incubation time and was expressed as log 2 value.

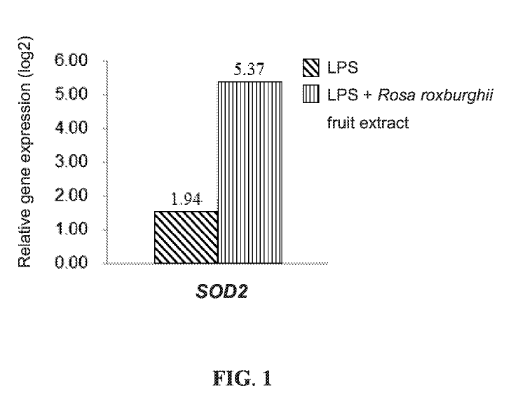

FIG. 1 shows the relative expression of SOD2 gene in LPS-stimulated THP-1 cells treated for 24 hours with or without the Rosa roxburghii fruit water extract as compared to the expression in control cells. According to FIG. 1, when compared to the THP-1 cells merely stimulated with LPS, the cells additionally treated with the Rosa roxburghii fruit extract showed significantly increased gene expression of SOD2. The result indicates that the Rosa roxburghii fruit extract enhances the ability of cells to remove reactive oxygen species and thus protects cells from damage by oxidative stress, such as those resulted from free radicals present in cells and the environment.

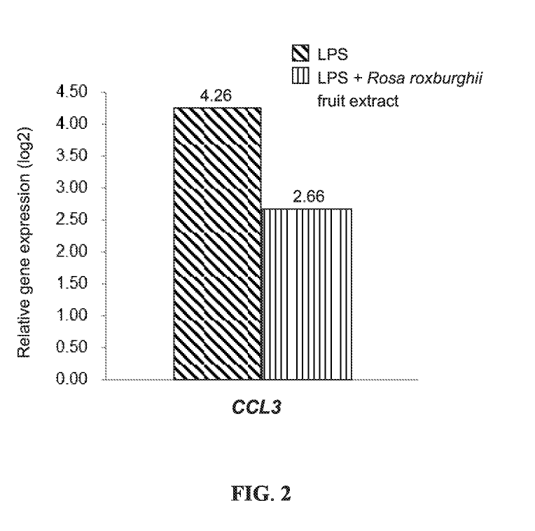

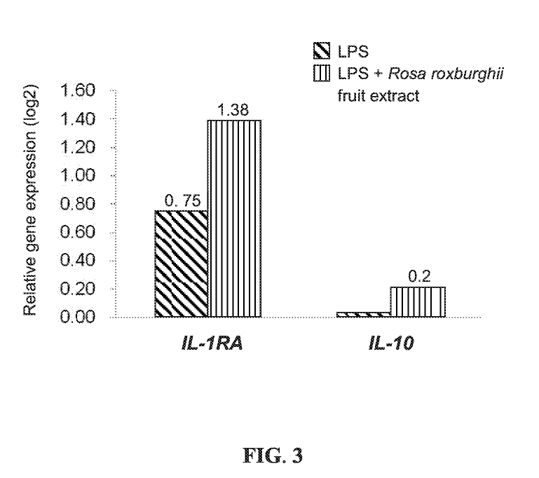

FIG. 2 shows the relative expression of CCL3 gene in LPS-stimulated THP-1 cells treated for 6 hours with or without the Rosa roxburghii fruit water extract as compared to the expression in control cells. FIG. 3 shows the relative expression of IL-1RA and IL-10 genes in LPS-stimulated THP-1 cells treated for 6 hours with or without the Rosa roxburghii fruit water extract as compared to the expression in control cells. According to FIG. 2 and FIG. 3, when compared to the THP-1 cells merely stimulated with LPS, the cells additionally treated with the Rosa roxburghii fruit extract showed significantly suppressed gene expression of the inflammation-associated CCL3, and exhibited elevated gene expression of the anti-inflammatory cytokines IL-1RA and IL-10. The results indicate that the Rosa roxburghii fruit extract inhibits the inflammatory response mediated by immune cells, thereby reducing cell damage by excessive inflammatory response in a subject.

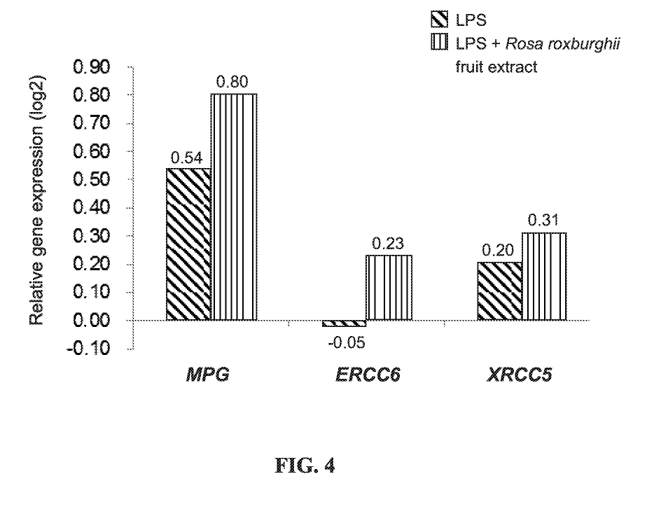

FIG. 4 shows the relative expression of MPG, ERCC6, and XRCC5 genes in LPS-stimulated THP-1 cells treated for 6 hours with or without the Rosa roxburghii fruit water extract as compared to the expression in control cells. According to FIG. 4, when compared to the THP-1 cells merely stimulated with LPS, the cells additionally treated with the Rosa roxburghii fruit extract showed significantly increased gene expression of the abovementioned three DNA repair proteins. The result indicates that the Rosa roxburghii fruit extract enhances the ability of cells to repair damaged DNA or genes and thus help maintain the normal function of genes.

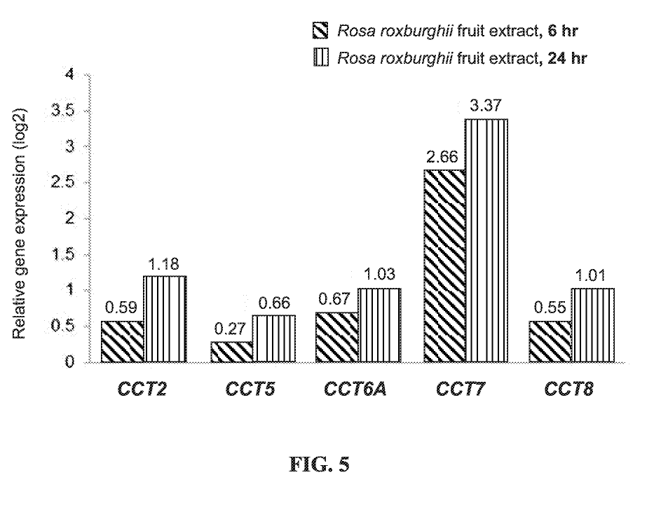

FIG. 5 shows the relative expression of CCT2, CCT5, CCT6A, CCT7, and CCT8 genes in THP-1 cells treated with the Rosa roxburghii fruit water extract for 6 or 24 hours as compared to the expression in control cells. According to FIG. 5, treatment with the Rosa roxburghii fruit extract enhanced the gene expression of the subunits of chaperonin containing TCP1 complex (CCT) in THP-1 cells, including the expression of CCT2, CCT5, CCT6A, CCT7, and CCT8 genes. This result shows that the Rosa roxburghii fruit extract increases the synthesis of chaperonin containing TCP1 complex that assists in protein folding, and is therefore beneficial for the normal structure and functioning of intracellular proteins.

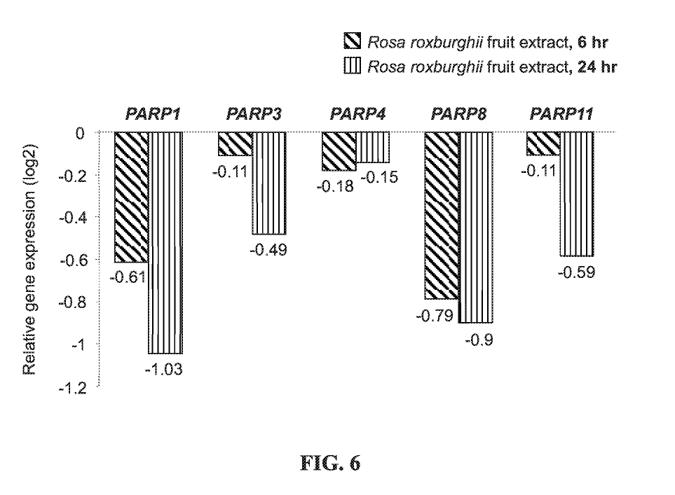

FIG. 6 shows the relative expression of the PARP1, PARP3, PARP4, PARP8, and PARP11 genes in THP-1 cells treated with the Rosa roxburghii fruit water extract for 6 or 24 hours as compared to the expression in control cells. According to FIG. 6, treatment with the Rosa roxburghii fruit extract inhibited the gene expression of various poly (ADP-ribose) polymerases (PARPs) in THP-1 cells. Since previous studies have pointed out that inhibition of this enzyme activity improves mitochondrial activity, this result shows that the Rosa roxburghii fruit extract enhances mitochondrial activity, thereby promoting cell energy production. Based on the experimental results in FIGS. 1-6, it is concluded that the Rosa roxburghii fruit extract is effective in rejuvenating the cells.

### Example 3

Enhancement of Expression of Genes Associated with Telomerase Reaction by Treatment of Rosa roxburghii Fruit Extract

Previous studies have revealed that increasing telomerase activity in cells promotes cell growth. In order to investigate the effect of Rosa roxburghii fruit extract on the expression of genes associated with telomerase reaction, qPCR was used to measure the changes in gene expression of telomerase reverse transcriptase (TERT) and telomerase ribonucleic acid component (TERC) in human monocytic cell line THP-1. First, THP-1 cells were seeded at 1.5×10⁵ cells/well in a 6-well plate, where each well contained 2 mL of RPMI cell culture medium. After culture at 37° C. for 24 hours, the cell culture medium was removed and the cells were washed with PBS. Then, cells in each well were treated with 500 μL of 2 mg/mL Rosa roxburghii fruit water extract and 500 μL of RPMI cell culture medium without FBS (experimental group), or treated only with 500 μL of RPMI cell culture medium without FBS (control group). The two groups of cells were subjected to qPCR analysis after incubated at 37° C. for 6 hours.

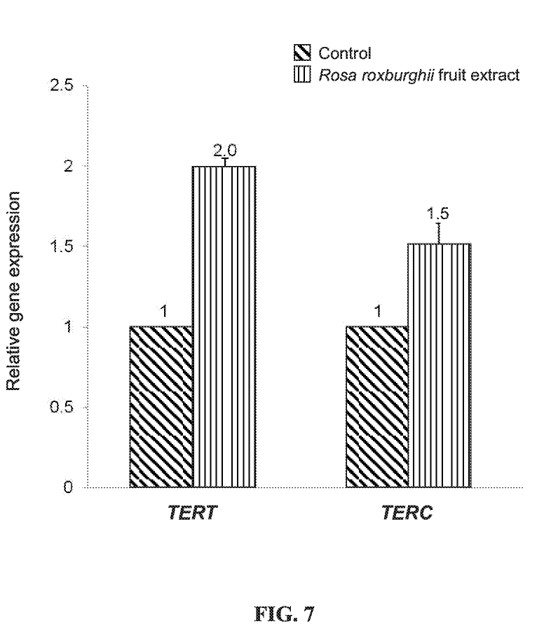

FIG. 7 shows the relative expression of TERT and TERC genes in THP-1 cells treated with the Rosa roxburghii fruit water extract for 6 hours or in control cells. The relative expression on the vertical axis of the figure indicates the fold change compared to the RNA expression of same gene in the control cells. According to FIG. 7, treatment of THP-1 cells with the Rosa roxburghii fruit extract resulted in a 2-fold and a 1.5-fold increase in gene expression of TERT and TERC, respectively. The result indicates that the Rosa roxburghii fruit extract is able to facilitate telomerase reaction in cells and telomere extension, and is effective in prolonging cell lifespan or delaying the aging process.

### Example 4

### Inhibition of Shortening of Chromosomal Telomere Length by Treatment of Rosa Roxburghii Fruit Extract

4.1 Protection from Chemical-Induced Telomere Shortening

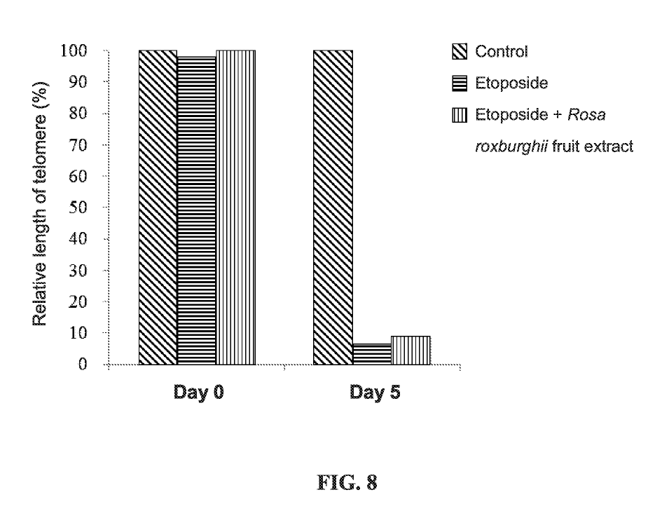

The protective effect of Rosa roxburghii fruit water extract on chromosomal telomere was investigated in human skin fibroblast CCD-966SK. First, CCD-966SK cells were seeded at 1.5×10⁵ cells/dish in three 35-mm culture dishes each containing 2 mL of MEM cell culture medium. After overnight culture at 37° C., half of the cells in each dish (day 0) were transferred into a RNA stabilization solution (RNA later solution; Ambion) and stored at −20° C. for subsequent use, and the remaining cells were separately seeded into another three 35-mm culture dishes and incubated at 37° C. overnight. Thereafter, cells in one of three dishes were treated with 30 μM etoposide (a chemotherapeutic agent). Cells in one of the other dishes were treated with 30 μM etoposide and 0.5 mg/mL of a Rosa roxburghii fruit water extract, and the remaining dish of cells was used as control and treated with no therapeutic agent and the Rosa roxburghii fruit water extract. After 5 days of incubation, the three groups of cells were collected (day 5). For telomere length determination by qPCR, genomic DNA was obtained from the aforementioned cells collected on day 0 and day 5 by using the Taco™ automatic nucleic acid extraction system (GeneReach Biotechnology) and the Taco™ DNA/RNA extraction kit. As shown in FIG. 8, when compared to the control cells, the cells treated only with etoposide had telomeres whose relative length was significantly shortened, whereas treatment with the Rosa roxburghii fruit water extract suppressed said telomere shortening, indicating that the Rosa roxburghii fruit extract help maintain the length of telomere that is damaged by chemicals.

4.2 Protection from Ultraviolet (UV) Irradiation-Induced Telomere Shortening

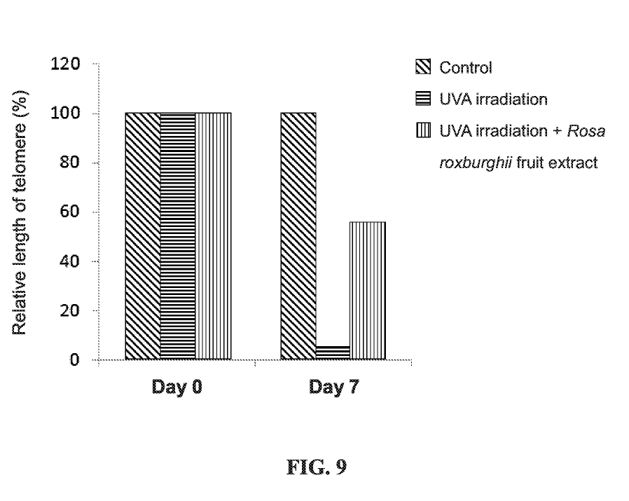

CCD-966SK cells were seeded at 1.5×10⁵ cells/dish in three 35-mm culture dishes each containing 2 mL of MEM cell culture medium. After overnight culture at 37° C., two dishes of cells were irradiated with 1 J/cm² UVA (wavelength 320-400 nm), while the remaining dish of cells was used as control and received no UVA radiation. Then, half of the cells in each dish (day 0) were transferred into a RNA stabilization solution (RNA later solution; Ambion) and stored at −20° C. for subsequent use, and the remaining cells were separately seeded into another three 35-mm culture dishes and incubated at 37° C. overnight. Thereafter, cells in one of the two dishes irradiated with UVA were treated with 0.5 mg/mL of a Rosa roxburghii fruit water extract, while the other dish of cells was not treated. After two subcultures during which the aforementioned ultraviolet irradiation was repeated each time, the three groups of cells were collected (day 7). For telomere length determination by qPCR, genomic DNA was obtained from the aforementioned cells collected on day 0 and day 5 by using the Taco™ automatic nucleic acid extraction system (GeneReach Biotechnology) and the Taco™ DNA/RNA extraction kit. As shown in FIG. 9, the when compared to the control cells, the cells irradiated with UVA had telomeres whose relative length was significantly shortened, whereas treatment with the Rosa roxburghii fruit water extract vastly reduced said telomere shortening, indicating that the Rosa roxburghii fruit extract help maintain the length of telomere that is damaged by ultraviolet light, In other words, the Rosa roxburghii fruit extract prevents photo-aging.

In conclusion, the above experiments have shown that the Rosa roxburghii fruit extract reduces cell damage caused by oxidative stress or inflammation, maintains the normal functions of genes, proteins, and mitochondria, facilitates telomerase reaction and reduces telomere damage by chemicals or UV radiation, ultimately leading to cell rejuvenation. Thus, the present invention provides a method for cell rejuvenation by contacting cells with a composition containing the Rosa roxburghii fruit extract. The composition is in the form of powders, granules, liquid, gel or paste, and is produced as foods, beverages, nutritional supplements, pharmaceuticals, or reagents that may be administered to a human subject via an oral or topical route.

The present invention has been described with reference to the above preferred embodiments. However, it will be apparent to those skilled in the art that modifications and changes in form and detail may be made without departing from the scope of the present invention defined by the appended claims.

What is claimed is:

1. A method for cell rejuvenation, comprising contacting a cell with a composition containing an effective amount of a Rosa roxburghii fruit extract obtained by extraction of a Rosa roxburghii fruit with a solvent.

2. The method of claim 1, wherein the solvent is water, alcohol, or a mixture of alcohol and water.

3. The method of claim 1, wherein the Rosa roxburghii fruit extract promotes telomerase synthesis.

4. The method of claim 3, wherein the Rosa roxburghii fruit extract enhances gene expression of telomerase reverse transcriptase (TERT), telomerase ribonucleic acid component (TERC), or combinations thereof.

5. The method of claim 1, wherein the Rosa roxburghii fruit extract inhibits chemical or UV irradiation-induced shortening of chromosomal telomere length.

6. The method of claim 1, wherein the Rosa roxburghii fruit extract improves gene repair.

7. The method of claim 6, wherein the Rosa roxburghii fruit extract enhances gene expression of a DNA repair protein selected from the group consisting of N-methylpurine DNA glycosylase (MPG), excision repair cross complementing 6 (ERCC6), X-ray repair cross complementing 5 (XRCC5), and any combination thereof.

8. The method of claim 1, wherein the Rosa roxburghii fruit extract promotes synthesis of chaperonin containing TCP1 complex (CCT).

9. The method of claim 8, wherein the Rosa roxburghii fruit extract enhances gene expression of a CCT subunit selected from the group consisting of CCT2, CCT5, CCT6A, CCT7, CCT8, and any combination thereof.

10. The method of claim 1, wherein the Rosa roxburghii fruit extract enhances mitochondrial activity.

11. The method of claim 10, wherein the Rosa roxburghii fruit extract inhibits gene expression of a poly (adenosine diphosphate-ribose) polymerase (PARP) selected from the group consisting of PARP1, PARP3, PARP4, PARP8, PARP11, and any combination thereof.

12. The method of claim 1, wherein the weight ratio of the solvent to the Rosa roxburghii fruit ranges from 20:1 to 1:1.

13. The method of claim 1, wherein the extraction is performed at a temperature between 50° C. and 100° C.

14. The method of claim 1, wherein the composition contains a water extract of Rosa roxburghii fruit at a concentration of at least 0.5 mg/mL.
